(19) United States
(12) Patent Application Publication
(10) Pub. No.: US 2021/0005288 A1
(43) Pub. Date: Jan. 7, 2021

(54) METHOD AND ARRANGEMENT FOR COLLECTING INPUT FROM A USER

(71) Applicant: CRF Box Oy, Helsinki (FI)

(72) Inventors: Antti Heikkilä, Helsinki (FI); Jerry Nygren, Helsinki (FI)

(21) Appl. No.: 16/872,529

(22) Filed: May 12, 2020

(30) Foreign Application Priority Data
Jul. 1, 2019 (FI) 20195597

(51) Int. Cl. G16H 10/20; G16H 10/60; G16H 15/00; G06K 9/00

(52) U.S. Cl. CPC G16H 10/20; G06K 9/00302; G16H 15/00; G16H 10/60

(57) ABSTRACT

Methods and devices are provided for collecting input information from a user. Prompts are presented to said user through a user interface to make said user provide input information. The input information is processed to provide data, which is transmitted through a telecommunications interface to one or more remote processing arrangements. Images are taken of a face of said user when input information was provided by said user through said user interface. Microexpressions are detected from said taken images, and information indicative of said microexpressions is derived and stored as mood information. The mood information stored in association with temporally separate instances of said user providing said input information is compared, and trend information is derived indicative of results of such comparing. Said trend information transmitted through said telecommunications interface.

### FIELD OF THE INVENTION

The invention is generally related to the technical field of collecting input from a user for later processing as a part of a larger campaign that must obey a strictly defined protocol. In particular the invention concerns the technology of automatically associating the collected input with information indicative of a level of engagement of the user.

### BACKGROUND OF THE INVENTION

Clinical trials constitute a large and well-established part of clinical studies. A clinical trial may be set up for example when the effects of a medical substance and/or other kind of treatment needs to be examined on a number of participants. Other types of clinical studies comprise for example observational studies that do not test potential treatments or substances but aim at developing new ideas about a disease or condition and its possible treatment. In order to give reliable results a clinical trial must follow a number of strictly defined principles involving aspects such as representativeness, regularity, duration, impartiality, accuracy, study design, participant eligibility, proper selection of study instruments according to phase of study, and the like. These principles are typically documented in the form of a clinical study protocol that is to be followed throughout the study. The alternative term clinical research protocol is also used.

FIG. 1 illustrates schematically some parties that may have a role in a clinical trial. The responsible party 101 who orders the clinical trial may be for example a research laboratory or a drug manufacturer. An organizing party 102 sets up the infrastructure 103 for collecting and transmitting data and takes care of e.g. proper anonymization of the collected data by acting as an interface between the responsible party 101 and the recruited participants 104. Clinical personnel 105 may be involved for example so that each participant 104 must pay regular visits to a monitoring physician during the period of executing the clinical trial. The participants 104 may also be called patients.

The trend in clinical trials has been towards increasing digitalization and virtual trials, in which independent action that the participants take at their own becomes more and more important. Some or all face-to-face interactions 106 between participants 104 and clinical personnel 105 may be replaced or augmented with digital information that is collected directly from the participants 104. Each participant may be given a user device, like a tablet computer, a smartphone, or similar, or they may utilize user devices of their own. The user device in question is configured with appropriate applications like electronic diary, data collection, and the like. The participants 104 are asked to regularly update the input that they give through their user devices, for example so that they take a test and/or respond to questions on the screen according to a given timetable that may also define times for taking the substance or other treatment that is being tested. The desired actions of participants, as well as the devices and applications used, are defined in the clinical study protocol.

Compliance is a general concept used to indicate the fidelity at which a particular user follows the prescribed instructions. A simple example of measuring compliance is to follow, whether the user answers each question on each given day. High compliance values are most desirable, because they can be interpreted to increase the reliability of the collected information. However, it has been found that compliance as such may not be enough to describe, whether the collected information is meaningful and reliable or whether each participant is actually behaving like he or she is expected to. As an example there may be considered a comparison between two fictitious participants A and B, both of which were observed to answer all questions 19 times out of 20. Thus they both achieved a compliance level of 95%. However, while participant A considered meticulously each question and ticked each answer box only after careful thinking, participant B just ran through the question list each time and gave the answers more or less by random. Such a situation can be described by saying that the level of engagement exhibited by A was much higher than that of B. In older kinds of clinical trials where data collection was through paper forms a low engagement level of a participant could take the form of the so-called parking lot syndrome, meaning that the participant filled in a number of neglected paper forms only at the very last minute while sitting in his or her car before coming in to the next face-to-face meeting.

Patient retention is a measure of how well the participants can be kept enrolled in the clinical trial so that they do not drop out while the clinical trial should be continuing. Getting someone recruited and properly initiated as a patient in a clinical trial may represent a significant investment, and if a patient drops out before the trial is completed all that money has been wasted. Patients that drop out may cause even bigger damage than just their direct share of the total investment, because the representativeness of the trial may degrade beyond the point after which the results are not considered reliable any more. Remote patient monitoring of some kind would be of advantage, particularly in highly digitalized clinical trials where human contacts with patients are scarce, if such monitoring can give indications of whether an intervention (i.e. a human contact) should be performed on a patient whose behavior may show early signs of possibly dropping out in the continuation.

Technical solutions that somehow enabled drawing conclusions of not only compliance but also the level of engagement of individual participants in performing their expected tasks would be highly welcome. As an example, the mere use of electronic user devices already makes it easier to detect whether a participant actually responded to questions, or otherwise used the trial application on the device, in the prescribed timetable and for a duration it takes to perform the task with proper consideration. However, not all problems of engagement monitoring can be solved this way. Participants have been found to be quite creative in hiding their cheating, for example by tampering with the internal time settings of their devices.

### SUMMARY

An objective of the invention is to present methods and arrangements for collecting input information from a user so that also such information can be obtained that enables evaluating aspects of engagement. Another objective of the invention is to enable collecting information that gives indications of possibly needed interventions in clinical trials. A further objective of the invention is to presents methods and embodiments that enable improving patient retention. A yet further objective of the invention is to present methods and embodiments of the kind above that are widely applicable in many kinds of clinical trials and applicable under a variety of clinical study protocols.

These and further advantageous objectives are achieved with a user device, a method, and a computer program product that enable detecting and utilizing microexpressions in association with input information collected from users, as described in the corresponding appended independent claims. Advantageous embodiments are described in the depending claims.

According to a first aspect there is provided a user device for collecting input information from a user. The user device comprises a user interface for presenting prompts to said user to make said user provide input information, a telecommunications interface for transmitting data to one or more remote processing arrangements, a controller coupled to said user interface and to said telecommunications interface for processing input information provided by said user through said user interface and for processing data to be transmitted through said telecommunications interface, and a camera coupled to said controller. The camera has an imaging sector. The controller is configured to receive images taken by said camera, and recognize microexpressions from images of human face taken when input information was provided by said user through said user interface, and derive and store information indicative or said microexpressions as mood information. The controller is configured to compare mood information stored in association with temporally separate instances of said user providing said input information, and derive trend information indicative of results of such comparing, and transmit said trend information through said telecommunications interface.

According to an embodiment said prompts comprise visual prompts; said user interface has a viewing sector within which the viewing of said visual prompts is possible; and said viewing sector at least partly overlaps with said imaging sector. This enables ensuring that the face of the user is available for taking images where microexpressions can be detected while the user views the prompts and provides corresponding input information.

According to an embodiment said controller is configured to transmit said input information provided by said user as raw data to at least one of said one or more remote processing arrangements. This involves the advantage that maximum fidelity to the original inputs given by the user is maintained also in later processing.

According to an embodiment said controller is configured to transmit said trend information through said telecommunications interface without associating it with any piece of said raw data. This involves the advantage that all unwanted bias in later processing of the raw data can be prevented.

According to an embodiment said controller is configured to transmit said trend information through said telecommunications interface in temporal association with respective pieces of said raw data. This involves the advantage that if needed, reliability of the raw data and its possible relations with emotional behavior of the user can be later analyzed if needed.

According to an embodiment said controller is configured to transmit said trend information through said telecommunications interface in strict piecewise association with pieces of said raw data, thus enabling associating individual pieces of said raw data with corresponding pieces of said trend information. This involves the advantage that if needed, reliability of the raw data and its possible relations with emotional behavior of the user can be later analyzed in detail if needed.

According to an embodiment said controller is configured to execute a recognition algorithm on said images of human face to recognize the user providing said input information, and only allow storing said input information in response to said recognition algorithm giving a match between the recognized user and a predetermined authorized user. This involves the advantage that features that serve the above explained purposes related to the utilization of microexpressions can be additionally used for enhanced data security.

According to a second aspect there is provided a method for collecting input information from a user. The method comprises presenting prompts to said user through a user interface to make said user provide input information, processing input information provided by said user through said user interface to provide data, and transmitting said data through a telecommunications interface to one or more remote processing arrangements. The method comprises taking images or a face of said user when input information was provided by said user through said user interface, recognizing microexpressions from said taken images, and deriving and storing information indicative of said microexpressions as mood information. The method comprises comparing mood information stored in association with temporally separate instances of said user providing said input information, and deriving trend information indicative of results of such comparing. The method comprises transmitting said trend information through said telecommunications interface.

According to an embodiment the method comprises presenting at least a part of said prompts as visual prompts in a viewing sector of said user interface, within which the viewing of said visual prompts is possible, which viewing sector at least partly overlaps with an imaging sector of a camera used for said taking of images. This enables ensuring that the face of the user is available for taking images where microexpressions can be detected while the user views the prompts and provides corresponding input information.

According to an embodiment said transmitting of data comprises transmitting said input information provided by said user as raw data to at least one of said one or more remote processing arrangements. This involves the advantage that maximum fidelity to the original inputs given by the user is maintained also in later processing.

According to an embodiment the method comprises transmitting said trend information through said telecommunications interface without associating it with any piece of said raw data. This involves the advantage that all unwanted bias in later processing of the raw data can be prevented.

According to an embodiment the method comprises transmitting said trend information through said telecommunications interface in temporal association with respective pieces of said raw data. This involves the advantage that if needed, reliability of the raw data and its possible relations with emotional behavior of the user can be later analyzed if needed.

According to an embodiment the method comprises transmitting said trend information through said telecommunications interface in strict piecewise association with pieces of said raw data, thus enabling associating individual pieces of said raw data with corresponding pieces of said trend information. This involves the advantage that if needed, reliability of the raw data and its possible relations with emotional behavior of the user can be later analyzed in detail if needed.

According to an embodiment the method comprises executing a recognition algorithm on said face of said user to recognize the user providing said input information, and only allowing storing said input information in response to said recognition algorithm giving a match between the recognized user and a predetermined authorized user. This involves the advantage that features that serve the above explained purposes related to the utilization of microexpressions can be additionally used for enhanced data security.

According to a third aspect there is provided a computer program product comprising, stored on a machine-readable medium, one or more sets of one or more machine-readable instructions that when executed on one or more processors are configured to cause the execution of a method of a kind explained above.

### BRIEF DESCRIPTION OF THE DRAWINGS

The accompanying drawings, which are included to provide a further understanding of the invention and constitute a part of this specification, illustrate embodiments of the invention and together with the description help to explain the principles of the invention. In the drawings:

FIG. 1 illustrates parties of a clinical trial,

FIG. 2 illustrates a user device,

FIG. 3 illustrates a method, and

FIG. 4 illustrates a method.

### DETAILED DESCRIPTION

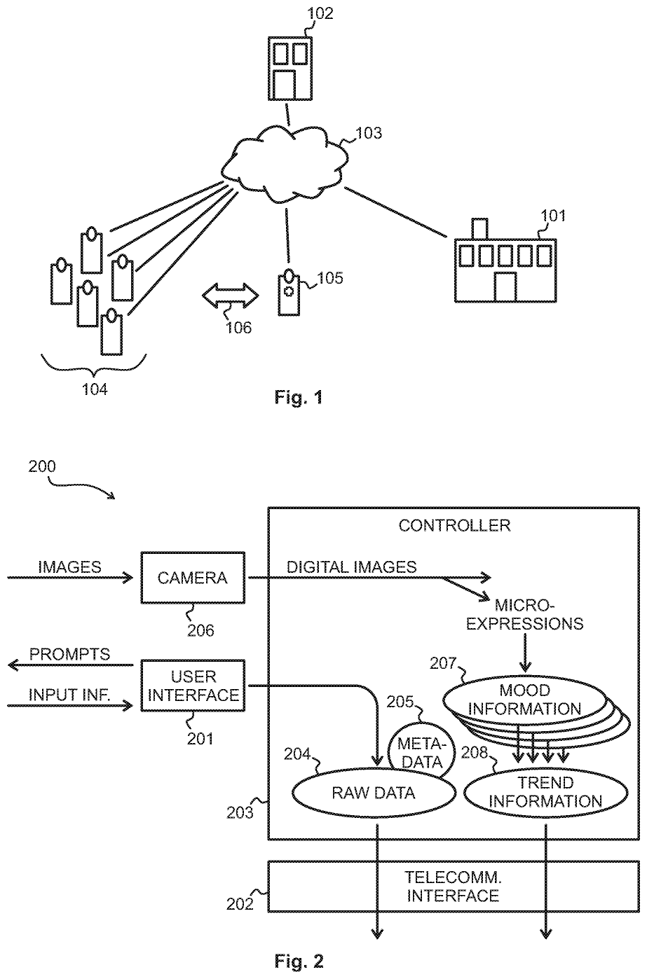

FIG. 2 illustrates schematically a user device 200 for collecting input information from a user. In particular, the user device 200 is meant for use in data collection as a part of a clinical trial, for the execution of which there is a clinical study protocol. The user device 200 comprises means for producing indications of the level of engagement of the user, as will be described in more detail in the following.

The user device comprises a user interface 201 for presenting prompts to the user. The user interface 201 may be, or may comprise, for example a display screen, the displayed contents of which can be defined by programming the user device 200 appropriately. The prompts may comprise visual prompts that can contain any kind of visually observable elements, such as text, other character strings, graphical symbols, color fields, images, selectable alternatives, input fields, and the like, as well as all combinations of these. The prompts need not necessarily consist of visually observable elements, but other kinds of elements can be used as well, such as audible elements reproduced by a suitable transducer included in the user interface 201. However, advantages can be gained in certain embodiments by using at least some visually observable elements that can be presented as prompts to the user through the user interface 201.

If at least some visually observable elements are used as visual prompts, the user interface 201 may have a viewing sector. The term viewing sector means the solid (i.e. three-dimensional) angle in which an observation point must be in order to conveniently see the displayed visually observable elements. As the prompts are to be presented to a human user, for such cases it can be defined that at least one eye of the user should be within the viewing sector of the user interface 201 for the user to conveniently see essential parts of the visual prompts.

The purpose of presenting prompts to the user is to prompt the user, i.e. to make the user provide input information to the user device 200 in return. In order to enable providing input information the user interface 201 should comprise also input means. A touch-sensitive display is an example of a type of a user interface hardware element that facilitates both visually presenting prompts to a user and the user providing input information to the user device 200. As an example, a visually presented prompt may comprise a question or statement displayed in text form. The corresponding input means may comprise images of keys, check boxes, radio buttons, or other kinds of fields that the user may touch as a sign of selecting one or more of the offered alternatives or to type in an answer in the form of a text string, a character indicating a selected point on a scale, or other. Other types of input means are for example hardware keys and buttons. The input means need not be tactile, but they may be or comprise other kinds of input means such as a microphone for recording responses that the user gives by speaking or making other noises.

The user device 200 comprises a telecommunications interface 202 for transmitting data to one or more remote processing arrangements. The hardware implementation of the telecommunications interface 202 is not important to this description. It may comprise either wired or wireless elements or both. For improving the convenience of using the user device 200 and for avoiding restrictions based on location it is advantageous if the telecommunications interface 202 is capable of conveying wireless communications, such as Wi-Fi communications and/or connections of mobile cellular networks for example. The telecommunications interface 202 may be for example similar to the telecommunications interfaces of smartphones and other portable communications devices meant for large-scale consumer use.

In order to transmit data to one or more remote processing arrangements in a controlled manner the user device is preferably configured to use one or more addressable transmission protocols, like the TCP/IP protocol for example. The remote processing arrangements may be for example servers or other computer devices coupled to a long-distance data communications network such as the internet. Since the participation in clinical trials may involve aspects of confidentiality concerning transmitted data, it is advantageous to configure the user device 200 to use confidentiality-enhancing measures in transmission, like data encryption protocols and/or VPN (Virtual Private Network) connections for example. Confidentiality-enhancing measures of this kind are routinely used in communications that involve aspects of confidentiality, and any of the known or future developed means can be used for the purpose described here.

The telecommunications interface 202 has been schematically shown as a single interface in FIG. 2. This is not a limiting feature: it may be advantageous to equip the user device 200 with interfaces to two or more completely different telecommunications systems. In this text when a reference is made to a telecommunications interface it may mean any or all possible parts through which the user device can communicate with other, remote devices.

Since an important purpose of utilizing the user device 200 is to make the user a participant in a clinical trial, the device should be configured for transmitting input information received from the user, or some information derived therefrom, to one or more remote processing arrangements related to the clinical trial. The user device comprises a controller 203 that is coupled to the user interface 201 and to the telecommunications interface 202, for processing input information provided by the user through the user interface 201, as well as for processing data to be transmitted through the telecommunications interface 202. The controller 203 may be or comprise one or more processors and/or microcontrollers, augmented with appropriate auxiliary circuitry such as memory circuits for storing data and computer-executable code.

Processing and transmitting in the way described above may be characterized as conveying information provided by a participant of the clinical trial to availability for the organizer of the clinical trial. One example of such conveying is i11ustrated in FIG. 2, where the controller 203 is configured to transmit the input information provided by the user as raw data 204 to at least one of the remote processing arrangements related to the clinical trial. Transmitting raw data 204, i.e. input information in exactly the way in which the user provided it to the user device 200, may be explicitly required by the clinical study protocol. The controller 203 may be configured to augment the raw data 204 with metadata 205 such as time stamps, location information, participant identification information, environmental variables (temperature, humidity, altitude, air pressure, speed, acceleration, etc.) or the like. At least some of the metadata 205 may come from components and/or internal processes of the user device, such as integrated environmental sensors or a rel time clock for example.

The user device 200 comprises a camera 206 that is coupled to the controller 203. The coupling is of a nature that allows images taken by the camera 206 to be conveyed to the controller 203 for processing in digital form. The coupling also preferably allows the controller 203 to control the camera 206 so that the controller 203 may decide, when the camera 206 is to obtain an image.

The camera 206 has an imaging sector that at least partly overlaps in space with the viewing sector of the user interface 201. The purpose of such overlap is to ensure that at least a part of the face of a human user who is actively looking at the user interface 201 is within the imaging sector of the camera 206. Sufficient overlap can be ensured through proper structural solutions of the user interface 201, the camera 206, and the user device 200 as a whole. One example of such structural solutions is the one frequently used in smartphones, tablets, and portable computers, in which the lens of at least one fixedly installed camera is located adjacent to a display, the main optical axes of the camera and display being essentially parallel to each other or so directed that they intersect at a convenient viewing distance such as about 40 cm from the display surface. Other structural solutions are possible, for example so that the lens of the camera 206 is located somewhere within a display that constitutes a part of the user interface 201, possibly hidden behind an unidirectionally reflective layer that keeps the user from seeing the camera.

For providing means for drawing conclusions of the level of engagement of the user, the camera 206 should be configured to take images of at least a part of the face of the user while the user is using the user interface 201 to provide input information related to the clinical trial. The controller 203 is configured to receive images taken by the camera 206, and to recognize microexpressions. Recognizing microexpressions can be augmented with interpreting, so that conclusions are made of recognized microexpressions in images taken by the camera 206.

Microexpressions are brief, involuntarily occurring facial expressions that face muscles produce in relation with emotions such as disgust, anger, fear, sadness, happiness, surprise, and contempt for example. A microexpression may occur very briefly, like during only a small fraction of a second. Image-analyzing algorithms are known and can be developed that receive digital images (or streams of images) of at least a part of a human face obtained with a camera and examine them for the occurrence of distinct microexpressions. Configuring the controller 203 to recognize microexpressions is most practical by storing such an image-analyzing algorithm in the form of machine-executable instructions in a program memory available in or for the controller 203. In particular, the controller 203 should be configured to run such an image-analyzing algorithm for images that were taken by the camera 206 when input information was provided by the user through the user interface 201.

Information about the possibly recognized microexpressions can be utilized to indicate aspects of engagement of the user. In an overly simplified form it might be expected that when the user is acting otherwise as he or she should as a participant of a clinical trial, but does not answer a question truthfully, a brief emotion of guilt for telling a lie may cause a microexpression to flash on his or her face. An image-analyzing algorithm may run in (or be accessible to) the controller 203, and analyze images that were obtained by the camera 206 at the same time when the user was giving the not completely truthful answer. Such an image-analyzing algorithm recognize the microexpression, and information indicative of this finding could be stored together with or otherwise linked to the answer that the user gave. Thus in this simplified example the user device would act as a kind of lie detector, enabling the storing of metadata 205 that would indicate that some of the input information provided by the user may be not completely reliable.

However, such an overly simplified example may not be the most advantageous way of applying the recognition of microexpressions, for a number of reasons. First, recognizing individual microexpressions and interpreting them correctly may not be very accurate or reliable, so the simplified example might lead to inaccurate or incorrect interpretations. Second, it may be against the principles expressed in the clinical study protocol to directly label any input information given by participants as not reliable or otherwise not as valuable as some other stored input information; the clinical study protocol may explicitly require storing the input information “as is”, without any further indications. Third, even if some input information provided by the user could be appropriately associated with some recognized simultaneous microexpression, it may be difficult to determine how that association should be taken into account in evaluating the input information, i.e. what consequences the recognition of the microexpression should imply.

It may be more advantageous to configure the controller 203 to derive and store information indicative of recognized microexpressions as mood information 207. Mood information 207 may be characterized as consisting of conclusions made on the basis of microexpressions that were recognized during an individual session of the user utilizing the user device 200 in the role of a participant of a clinical trial. Such a session may be for example the duration of time when the user answers a set of questions allocated for responding on a particular day. The duration of a session may be defined for example as the time from opening the clinical trial application to answering the last question of the day, to closing the application, or to otherwise indicating that the session was completed.

Thus the mood information 207 is, as its name says, information about the mood that the user was in at the time of providing the input information, in light of what was revealed by his or her recognized microexpressions. For example, if the controller 203 is configured to recognize at most a fixed number or microexpression types 1, 2, . . . , N, mood information 207 stored for one session may comprise a list of how many times there was recognized a microexpression of type 1, how many times a microexpression of type 2, and so on. As another example, mood information 207 stored for one session may comprise an indicator of what was the most frequently recognized microexpression curing that session. As yet another example, mood information 207 stored for one session may comprise information about the frequency per unit time of recognizing any microexpressions during that session. As yet another example, mood information 207 stored for one session may comprise one or more indicators of now the recognized microexpressions developed during that session (for example: at the beginning of session there was detected a majority of happy microexpressions, while towards the end of the session more and more microexpressions of contempt began to appear).

A particularly valuable aspect of mood information may be not what it tells about an individual session but what it reveals as a change over time. The controller 203 may be configured to compare mood information 207 stored in association with temporally separate instances (i.e. sessions) of the user providing input information, and to derive trend information 208 indicative of results of such comparing. The controller 203 may be configured to transmit the trend information so obtained through the telecommunications interface 202 to an appropriate recipient, like one of the one or more remote processing arrangements associated with the clinical trial.

In comparing mood information 207 that was stored in association with temporally separate instances, the controller 203 may focus the comparisons on the basis of the questions or types of questions. Prompts or questions can be said to represent a number of types, such as pleasant, unpleasant, easy, difficult, superficial, deep, motivating, frustrating, emollient, irritating, and so on. Same questions may repeat from session to session, in which case it may be advantageous to compare mood information stored in association with the moments during temporally separate instances when a particular question occurred. Additionally or alternatively there may be compared mood information stored in association with the moments during temporally separate instances when questions of particular type occurred. The comparison may take into account some natural developments, like the natural tendency of a participant to grow bored of answering the same question again and again; mood information that indicates gradually increasing jadedness may be considered natural, while signs of additional aggressiveness may be considered exceptional or alerting.

According to one advantageous embodiment an organizer of the clinical trial (or a party otherwise responsible for at least part of the proceeding of the clinical trial) may utilize the trend information 208 received from user devices to evaluate, which participants should be called in to face-to-face meetings (i.e. interventions) and when participants from which raw data 204 has been received regularly, and for which their associated trend information shows little or no change in what their recognized microexpressions tell from one session to another, may not be in immediate need of a face-to-face meeting. On the other hand if from a certain participant raw data 204 has been received regularly but the trend information 208 shows a worrisome trend in moods, like an exceptional increase in the occurrence of recognized microexpressions associated with negative emotions, a call to a face-to-face meeting may be appropriate to find out the reason behind the development. Such use of the trend information 208 may be augmented with using other stored metadata, such as environmental observations that were stored in association with the mood information 207 and/or the trend information 208, for example so that an increase in microexpressions related to anxiety is not considered quite as alarming if it was associated with simultaneous occurrence of thunderstorms or other severe weather conditions.

For reasons described above in association with the clinical study protocol it may be advantageous to configure the controller 203 to transmit the trend information 208 through the telecommunications interface 202 without associating it with any particular piece of the raw data 204. This way no bias is introduced into the raw data, which consequently represents as accurately and unbiased as possible what the user actually has provided as input reformation. The raw data 204 and trend information 208 may be transmitted to different remote processing arrangements to begin with, or the remote processing arrangement to which they are initially transmitted may separate them and anonymize them from each other so that it is not possible to tell later, which piece of raw data would correspond to what trend information.

As an alternative, the controller 203 may be configured to transmit the trend information 208 through the telecommunications interface in temporal association with respective pieces of the raw data 204. This can be accomplished in a number of ways. For example, each time when the controller 203 transmits raw data it may transmit corresponding trend information simultaneously or essentially simultaneously, like during the same minute, the same hour, or the same day. The remote processing, arrangement(s) receiving such raw data and trend information can then at least correlate the moments of receiving raw data and trend information to find out their temporal association. Another way of achieving temporal association is to augment each piece of transmitted raw data and trend information with a corresponding time stamp, so that the remote processing arrangement(s) receiving such time-stamped raw data and trend information can find out the temporal association by comparing the time stamps.

In some cases it may be even desirable that trend information, or even mood information, can be later associated directly with particular pieces or raw data. In such cases the controller 203 may be configured to transmit the trend information 208 (or mood information 207) through the telecommunications interface 202 in strict piecewise association with pieces of the raw data 204, thus enabling associating individual pieces of raw data with corresponding, pieces of trend information.

The capability of the user device 200 to obtain and process images of at least a part of the face of the user may be utilized also for other purposes than for recognizing microexpressions. One aspect of clinical trials is the strict requirement of being just the desired participant and not someone else providing the input information. The controller 203 may be configured to execute a recognition algorithm on the images of human faces it receives from the camera 206, to biometrically recognize the user providing the input information. The controller 203 may be configured to only allow storing the input information in response to the recognition algorithm giving a match between the recognized user and a predetermined authorized user.

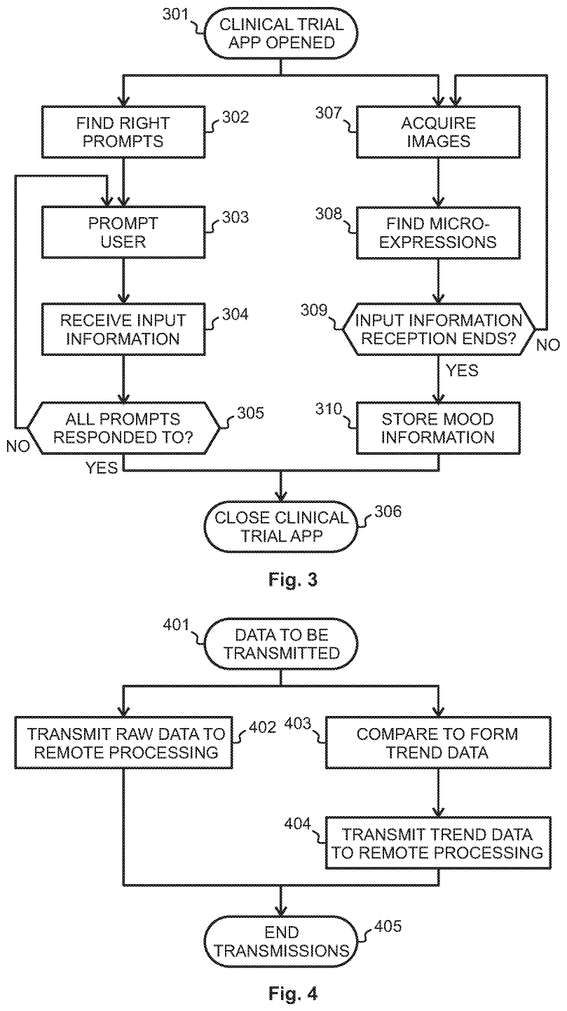

FIG. 3 illustrates a method that the user device may execute in the form of a flow diagram. A session during which the user acts as a participant of the clinical trial begins when the user opens the clinical trial app on the user device at his or her disposal (step 301). Opening the app triggers two parallel processing branches. In the left branch the app checks for defining data such as date, time, log of previously stored data and/or others, and finds the prompts that are currently actual for presenting to the user at step 302. Processing in the left branch proceeds in a loop through prompting the user at step 303, receiving input information at step 304, and checking whether all actual prompts has been responded to at step 305, until a positive finding at step 305 allows ending the session and closing the app at step 306.

Simultaneously in the right branch the camera takes images of the user at step 307, and these images are analyzed to find microexpressions at step 308. Step 309 is a check whether processing in the left branch has reached a positive finding at step 305. If not, the process of acquiring images and looking for microexpressions continues. A positive finding at step 309 leads to deriving and storing information indicative of the recognized microexpressions as mood information at step 310, after which the clinical trial app can be closed also with respect to the right branch at step 306.

Transmitting to the one or more remote processing arrangements is shown separately as a method in FIG. 4. It is typically not necessary to transmit data from the user device immediately after it has been acquired, although that is not excluded either. In FIG. 4 it is assumed that when there is at least some data to be transmitted and the situation is also otherwise favorable, for example so that a reasonably priced communications connection is available, transmission begins at step 401. If there is input information provided by the user that has not yet been reported to the remote processing arrangement, a corresponding transmission is made at step 402. In FIG. 4, similar to FIG. 2, it is assumed that the input information provided by the user is transmitted as raw data to at least one of the one or more remote processing arrangements.

On the right in FIG. 4, step 403 corresponds to comparing mood information stored in association with temporally separate instances of the user providing input information, and deriving trend information indicative of results of such comparing. Step 403 can also have been performed earlier, because comparing of this kind can be made whenever there is mood information to be compared and sufficient free processing capacity available in the user device. Step 404 corresponds to transmitting the trend information through the telecommunications interface to at least one of the one or more remote processing arrangements. Transmission ends at step 405 when all actual data to be transmitted has been transmitted.

It is obvious to a person skilled in the art that with the advancement of technology, the basic idea of the invention may be implemented in various ways. The invention and its embodiments are thus not limited to the examples described above, instead they may vary within the scope of the claims.

1. User device for collecting input information from a user, the device comprising:
a user interface for presenting prompts to said user to make said user provide input information;
a telecommunications interface for transmitting data to one or more remote processing arrangements;
a camera having an imaging sector; and
a controller coupled to said user interface, said camera, and to said telecommunications interface, said controller including a processor for processing input information provided by said user through said user interface and for processing data to be transmitted through said telecommunications interface, said controller further including memory having instructions stored thereon, which when executed by said processor, cause said device to
receive images taken by said camera of a human face when input information was provided by said user through said user interface, and with said processor, convert said received images into image data including microexpression data indicative of microexpressions of said human face and non-microexpression data;
process said image data with said processor to separate said microexpression data from said non-microexpression data and determine mood information based on said microexpression data;
store mood information on said memory;
with said processor, compare said stored mood information with stored data of mood information of temporally separate instances of said user providing said input information, and derive trend information indicative of results of such comparing, said trend information indicating a change of mood information of the human face over time;
augment said trend information with metadata stored on said memory to improve accuracy of said trend information; and
transmit said augmented trend information through said telecommunications interface.

2. A user device according to claim 1, wherein:
said prompts comprise visual prompts,
said user interface has a viewing sector within which the viewing of said visual prompts is possible, and
said viewing sector at least partly overlaps with said imaging sector.

3. A user device according to claim 1, wherein said controller is configured to:
transmit said input information provided by said user as raw data to at least one of said one or more remote processing arrangements.

4. A user device according to claim 3, wherein said controller is configured to:
transmit said trend information through said telecommunications interface without associating it with any piece of said raw data.

5. A user device according to claim 3, wherein said controller is configured to:
transmit said trend information through said telecommunications interface in temporal association with respective pieces of said raw data.

6. A user device according to claim 3, wherein said controller is configured to:
transmit said trend information through said telecommunications interface in strict piecewise association with pieces of said raw data, thus enabling associating individual pieces of said raw data with corresponding pieces of said trend information.

7. A user device according to claim 1, wherein said controller is configured to:
execute a recognition algorithm on said images of human face to recognize the user providing said input information, and
only allow storing said input information in response to said recognition algorithm giving a match between the recognized user and a predetermined authorized user.

8. Method for collecting input information from a user, the method comprising:
presenting prompts to said user through a user interface to make said user provide input information,
with a processor, processing input information provided by said user through said user interface to provide data,
transmitting said data through a telecommunications interface to one or more remote processing arrangements,
with a camera, taking images of a face of said user when input information is provided by said user through said user interface,
converting images into image data including microexpression data indicative of microexpressions of said face and non-microexpression data,
processing said microexpression data with said processor to separate said microexpression data from said non-microexpression data and to determine mood information based on said microexpression data,
storing determined mood information on a memory operably coupled to said processor,
with said processor, comparing said stored determined mood information with stored data of mood information of temporally separate instances of said user providing said input information, and deriving trend information indicative of results of such comparing, said trend information indicating a change of mood information of said face over time,
with said processor, processing said trend information with metadata stored on said memory to augment said trend information;
transmitting said augmented trend information through said telecommunications interface.

9. A method according to claim 8, comprising:
presenting at least a part of said prompts as visual prompts in a viewing sector of said user interface, within which the viewing of said visual prompts is possible, which viewing sector at least partly overlaps with an imaging sector of a camera used for said taking of images.

10. A method according to claim 8, wherein said transmitting of data comprises transmitting said input information provided by said user as raw data to at least one of said one or more remote processing arrangements.

11. A method according to claim 10, comprising:
transmitting said trend information through said telecommunications interface without associating it with any piece of said raw data.

12. A method according to claim 10, comprising:
transmitting said trend information through said telecommunications interface in temporal association with respective pieces of said raw data.

13. A method according to claim 8, comprising:
transmitting said trend information through said telecommunications interface in strict piecewise association with pieces of said raw data, thus enabling associating individual pieces of said raw data with corresponding pieces of said trend information.

14. A method according to claim 8, comprising:
executing a recognition algorithm on said face of said user to recognize the user providing said input information, and
only allowing storing said input information in response to said recognition algorithm giving a match between the recognized user and a predetermined authorized user.

15. A non-transitory computer program product comprising, stored on a machine-readable medium, one or more sets of one or more machine-readable instructions that when executed on one or more processors are configured to cause the execution of a method that comprises:
presenting prompts to said user through a user interface to make said user provide input information,
with a processor, processing input information provided by said user through said user interface to provide data,
transmitting said data through a telecommunications interface to one or more remote processing arrangements,
with a camera, taking images of a face of said user when input information is provided by said user through said user interface,
converting images into image data including microexpression data indicative of microexpressions of said face and non-microexpression data,
processing said microexpression data with said processor to separate said microexpression data from said non-microexpression data and to determine mood information based on said microexpression data,
storing determined mood information on a memory operably coupled to said processor,
with said processor, comparing said stored determined mood information stored in association with stored data of mood information of temporally separate instances of said user providing said input information, and deriving trend information indicative of results of such comparing, said trend information indicating a change of mood information of said face over time,
with said processor, processing said trend information with metadata stored on said memory to augment said trend information; and
transmitting said augmented trend information through said telecommunications interface.
